(19) United States
(12) Patent Application Publication
(10) Pub. No.: US 2023/0017792 A1
(43) Pub. Date: Jan. 19, 2023

(54) DRIVELINE/CONNECTOR FOR USE WITH IMPLANTABLE HVAD PUMP OR LVAS SYSTEMS

(71) Applicant: Medtronic, Inc., Minneapolis, MN (US)

(72) Inventors: Kevin Seifert, Forest Lake, MN (US); Jonathan A. Hughes, Blaine, MN (US); Andrei Draghici, Hallandale Beach, FL (US); Lisa A. Meyer, Faribault, MN (US); Andrew J. Ries, Lino Lakes, MN (US); Thomas J. Fick, Minneapolis, MN (US)

(21) Appl. No.: 17/378,067

(22) Filed: Jul. 16, 2021

(51) Int. Cl. A61M 60/878; H01R 24/58; H01R 13/22; H01R 13/41

(52) U.S. Cl. CPC A61M 60/878; H01R 24/58; H01R 13/22; H01R 13/41; A61M 60/178

(57) ABSTRACT

A connector for a medical device. The connector including an elongate body having a proximal end and a distal end. The connector further including a strut, a plurality of conductors, and a plurality of electrical contacts. The strut being disposed within the elongate body and defining a plurality of channels. Each of the plurality of channels having a proximal end and a distal end opposite the proximal end. The plurality of conductors being disposed within the elongate body and are sized and configured to be received within the plurality of channels and extend between the proximal end and the distal end. The plurality of electrical contacts being coupled to an outer surface of the elongate body and in communication with the plurality of conductors.

### CROSS-REFERENCE TO RELATED APPLICATION

n/a.

### FIELD

The present technology is generally related to implantable medical devices and transcutaneous energy transfer systems (TETS).

### BACKGROUND

Current ventricular assist device (VAD) systems utilize single multi-pin connectors that are designed for pacing and defibrillation leads/systems. Known connectors are not fully implantable within a living organism and are not configured to handle higher continuous energies in a wet environment.

### SUMMARY

The techniques of this disclosure generally relate to a connector for an implantable medical device.

In one aspect, the present disclosure provides a connector for a medical device, comprising an elongate body having a proximal end and a distal end, and a strut disposed within the elongate body. The strut defining a plurality of channels each having a proximal end and a distal end opposite the proximal end. The elongate body further includes a plurality of conductors disposed within the elongate body. The plurality of conductors being sized and configured to be received within the plurality of channels and extend between the proximal end and the distal end of the elongate body. The elongate body further includes a plurality of electrical contacts coupled to an outer surface of the elongate body. The plurality of electrical contacts being in communication with the plurality of conductors.

In another aspect, the elongate body further defines a plurality of grooves, each of the plurality of electrical contacts are aligned and retained within a respective groove of the plurality of grooves.

In another aspect, the plurality of electrical contacts each have an inner portion and an outer portion opposite the inner portion, the inner portion defines an aperture sized to receive a distal end of one of the plurality of channels.

In another aspect, each channel of the plurality of channels progressively decreases in length between a longest channel of the plurality of channels and a shortest channel of the plurality of channels.

In another aspect, each of the plurality of electrical contacts are ring contacts.

In another aspect, the strut is made out of a first electrically insulative material.

In another aspect, the connector further includes a non-conductive sealing surface disposed within the elongate body between the proximal end and the distal end.

In another aspect, the plurality of electrical contacts includes a first set of electrical contacts and a second set of electrical contacts, the non-conductive sealing surface being positioned between the first and second sets of electrical contacts to electrically isolate each respective set from one another.

In another aspect, the elongate body has an outer diameter between the range of 0.160 to 0.250 inches.

In another aspect, the elongate body defines a longitudinal axis between the proximal end and the distal end, each of the plurality of electrical contacts are spaced apart from an adjacent electrical contact along the longitudinal axis.

In another aspect, the elongate body is made up of a second electrically insulative material selected from the group consisting of polyurethane, polyethylene, urethane, and polyether ether ketone (PEEK).

In yet another aspect, a connector for a medical device includes a first elongate body and a second elongate body parallel to the first elongate body. The first elongate body and the second elongate body each have a proximal end and a distal end opposite the proximal end. The connector further includes a first plurality of conductors disposed within the first elongate body, a second plurality of conductors disposed within the second elongate body, a first plurality of electrical contacts extending around an outer surface of the first elongate body, and a second plurality of electrical contacts extending around an outer surface of the second elongate body. The first plurality of conductors are in communication with the first plurality of electrical contacts. The second plurality of conductors are in communication with the second plurality of electrical contacts.

In another aspect, the first plurality of conductors includes more conductors than the second plurality of conductors.

In another aspect, the first and second plurality of conductors each include at least two conductors.

In another aspect, the first and second plurality of electrical contacts each include at least two electrical contacts.

In another aspect, each electrical contact of the first and second plurality of electrical contacts is a ring contact.

In another aspect, the first elongate body and the second elongate body each define a longitudinal axis between the proximal end and the distal end, each ring contact is spaced apart from an adjacent ring contact along the longitudinal axis of the first elongate body and the second elongate body respectively.

In another aspect, the connector further includes a non-conductive sealing surface disposed within at least one selected from the group consisting of the first elongate body and the second elongate body.

In another aspect, the first elongate body and the second elongate body are each made up of an electrically insulative material.

In yet another aspect, a connector for a medical device includes an electrically insulative elongate body having an outer diameter between the range of 0.160 to 0.250 inches. The electrically insulative elongate body includes a proximal end, a distal end opposite the proximal end, a longitudinal axis defined between the proximal end and the distal end, and a plurality of grooves defined by an outer surface of the electrically insulative elongate body along the longitudinal axis. The connector further includes a strut disposed within the electrically insulative elongate body. The strut defines a plurality of channels each having a proximal end and a distal end opposite the proximal end. Each channel of the plurality of channels progressively decreases in length between a longest channel of the plurality of channels and a shortest channel of the plurality of channels. The connector further includes a plurality of conductors sized and configured to be received within the plurality of channels. The plurality of conductors include a first set of conductors and a second set of conductors. The connector further includes a plurality of electrical ring contacts extending around the outer surface of the electrically insulative elongate body. Each of the plurality of electrical ring contacts is aligned and retained within a respective groove of the plurality of grooves and has an inner portion and an outer portion opposite the inner portion. The inner portion defines an aperture sized to receive a distal end of one conductor of the plurality of conductors. The connector further includes a non-conductive sealing surface disposed between the proximal end and the distal end of the elongate body. The non-conductive sealing surface being configured to electrically isolate the first set of conductors from the second set of conductors.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an internal system view of an implantable blood pump with a TETS receiver source constructed in accordance with the principles of the present application;

FIG. 2 is a side view of a first embodiment of a connector for use with the medical systems shown in FIG. 1;

FIG. 3 is an exploded view illustrating the connection between the connector of FIG. 2 and a corresponding header;

FIG. 4 is a sectional view illustrating the cross-section A-A of the header and the internal alignment of the connector of FIGS. 3-4 when fully inserted into the header;

FIG. 5 is a side view of a strut disposed within the connector shown in FIG. 3;

FIG. 6 is a perspective view illustrating the engagement of a plurality of channels of the strut of FIG. 5 and a plurality of electrical contacts;

FIG. 7 is an exploded view showing the engagement of a plurality of conductors with the plurality of channels; and

FIG. 8 is a perspective view of a second embodiment of the connector of FIGS. 1-8.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

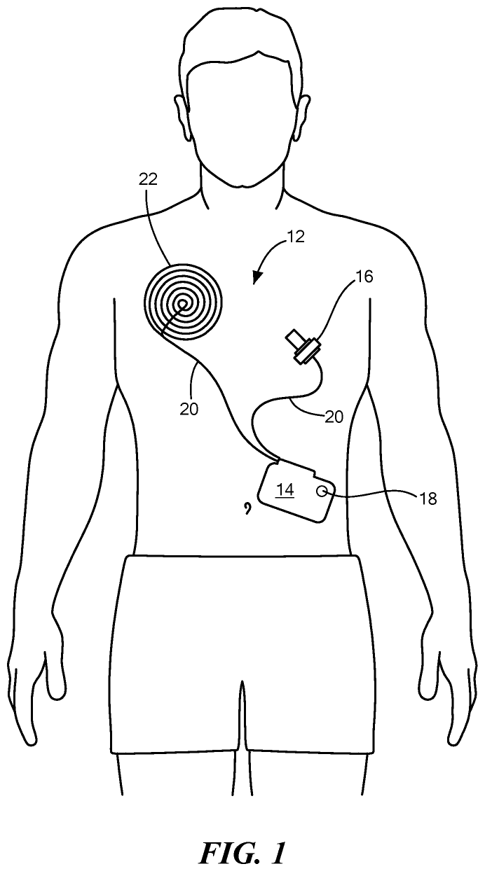

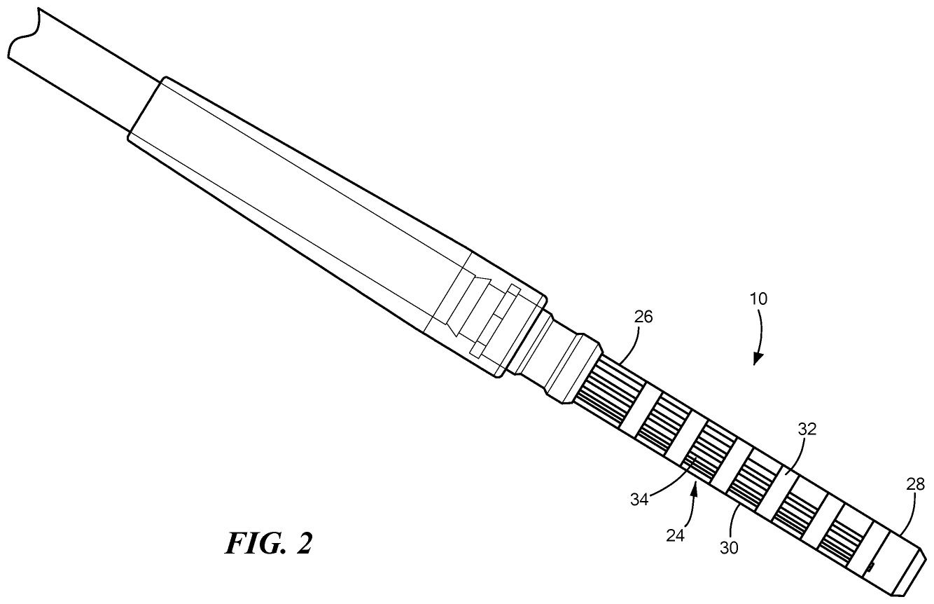

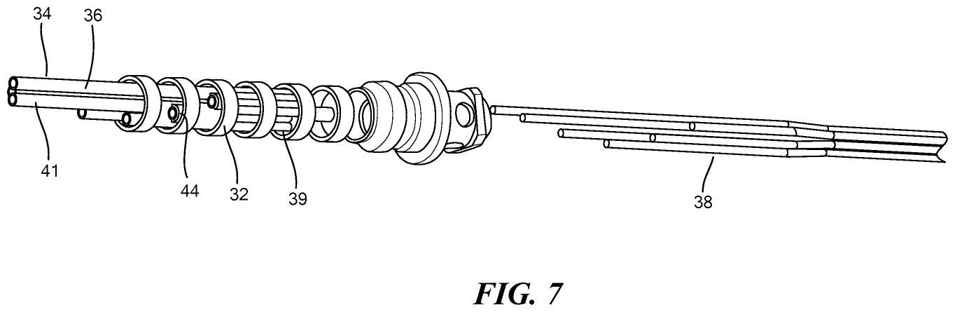

Referring now to FIGS. 1-8, the drawings in which like reference designators refer to like elements there is shown in FIGS. 1 and 2 an exemplary connector 10 for a mechanical circulatory support device (“MCSD”) 12 constructed in accordance with the principles of the present application. The connector 10 and MCSD 12 may be partially or fully implantable within a patient, whether human or animal, which is to say in one embodiment there may be percutaneous connectors between the implanted components of the connector 10 and the components outside of the body of the patient, and in another embodiment, there may be no percutaneous connections between the implanted components of the connector 10 and the components outside of the body of the patient. In the configuration shown in FIG. 1, the MCSD 12 includes an internal controller 14 implanted within the body of the patient. The internal controller 14 includes a control circuit having processing circuitry configured to control operation of an implantable blood pump 16. The internal controller 14 may include an internal power source, configured to power the components of the controller 14 and provide power to one or more implantable medical devices, for example, the implantable blood pump 16, such as a ventricular assist device (“VAD”) implanted within the left ventricle of the patient's heart. The power source may include a variety of different types of power sources including an implantable battery 18. VADs may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No. 8,419,609, the entirety of which is incorporated herein by reference. In an exemplary configuration, the blood pump 16 is electrically coupled to the internal controller 14 by one or more implanted cables 20 via the connector 10. The implanted cables 20 are configured to provide power to the pump 16, relay one or more measured feedback signals from the pump 16, and/or provide operating instructions to the pump 16. The connector 10 may facilitate the use of other existing medical systems such as the Medtronic Attain® Family of Inline Leads and the Medtronic Sprint Quattro Secure™ model leads.

Continuing to refer to FIG. 1, a receiving or internal coil 22 may also be coupled to the internal controller 14 by, for example, the one or more implanted cables 20. In an exemplary configuration, the receiving coil 22 may be implanted subcutaneously proximate the thoracic cavity, although any subcutaneous position may be utilized for implanting the receiving coil 22. The receiving coil 22 is configured to be inductively powered through the patient's skin by a transmission or external coil (not shown) disposed opposite the receiving coil 22 on the outside/exterior of the patient's body. The receiving coil 22 may be configured for transcutaneous inductive communication with the transmission coil to define a transcutaneous energy transfer system (TETS) that receives power from the transmission coil.

Referring now to FIGS. 2-4, the connector 10 includes an elongate body 24 having a proximal end 26 and an opposite distal end 28, and defining a longitudinal axis therebetween. The elongate body 24 also has an inner surface (not shown) and an opposite outer surface 30 extending laterally between the proximal end 26 and the distal end 28. The elongate body 24 is composed of an electrically insulative and rigid material such as polyurethane, polyethylene, polyether ether ketone (PEEK), silicone, urethane, and rubber, that is used to protect the patient from electrical current transmitted through the connector 10 once implanted within the patient's body.

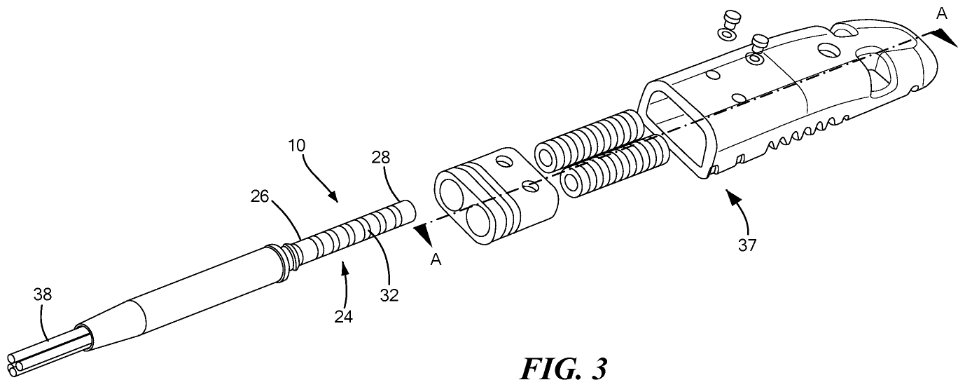

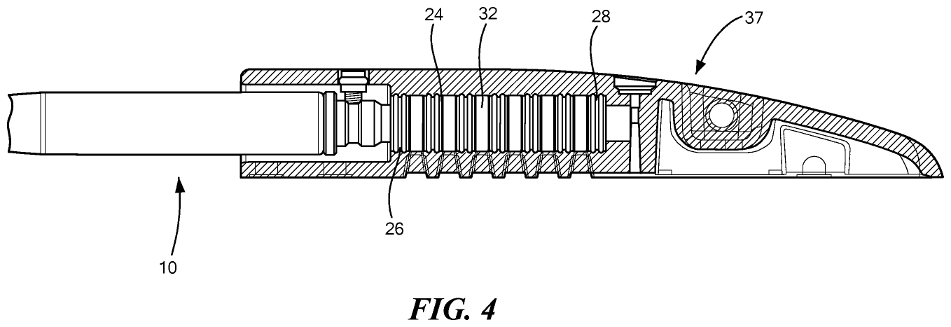

The connector 10 includes a plurality of electrical contacts 32, a strut 34 having a plurality of channels 36, and a plurality of electrical conductors 38 configured to be received within a respective channel 36 of the strut 34. The conductors 38 are partially disposed within the cables 20 extending between the connector 10 and the pump 16. As discussed in more detail below, the elongate body 24 is formed such that it defines intermediate portions between each of the contacts 32 to allow for physical and electrical separation of the contacts 32. The elongate body 24 is formed between each electrical contact 32 so that the electrical contacts 32 may engage a corresponding header such as, for example, the header 37 shown in FIGS. 3 and 4. Additionally, the elongate body 24 may define an outer diameter between the range of 0.130 inches to 0.250 inches. However, in one exemplary embodiment, the elongate body 24 defines an outer diameter between the range of 0.160 inches to 0.250 inches. This diameter range allows for increased isolation between the components disposed within the elongate body 24 and allows for easier assembly and molding of corresponding mating members (e.g., corresponding connector headers). The diameter and surface area of the elongate body 24 also provides more contact points to reduce electrical resistance and improve rigidity of the connector 10, which allows for increased insertion force for additional contact and seals when connecting the connector 10 into a corresponding header.

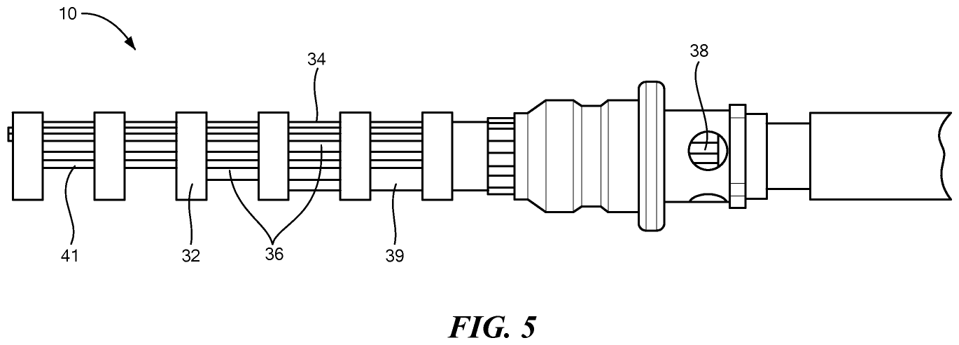

As shown in more detail in FIGS. 5-7, the strut 34 defines the plurality of channels 36 which are sized and configured to allow for the passage of the conductors 38. Each channel of the plurality of channels 36 has a proximal end 39 and a distal end 41 opposite the proximal end 39. Each channel 36 may be composed of a material such as medical grade polyurethane, polyethylene, PEEK, epoxy, or other types of casting or molded materials, and provides controlled separation and electrical isolation for each conductor 38 from the other adjacent conductors 38 disposed within the elongate body 24. In one embodiment, the strut 34 may have a staircase-like configuration so that each channel 36 has a different length. In this configuration, the length of a particular channel 36 may extend distally or progressively beyond the distal end 41 of an adjacent channel 36 towards the distal end 28 of the connector 10. In other words, each channel 36 of the plurality of channels 36 may progressively decrease in length between a longest channel 36 of the plurality of channels 36 and a shortest channel 36 of the plurality of channels 36. Each channel may also be in physical contact with at least one adjacent channel 36, or partially spaced apart from an adjacent channel 36. For example, each channel 36 may include an outer wall that is partially joined to an outer wall of an adjacent channel 36 and allows for a more streamlined configured that is sized to be retained within the elongate body 24.

Continuing to refer to FIGS. 5-7, the plurality of electrical contacts 32 extend at least partially or fully around the outer surface 30 or circumference of the elongate body 24 and are electrically coupled to the plurality of electrical conductors 38. Additionally, as a result of the overmolding process, in one embodiment, the plurality of electrical contacts 32 are aligned and retained within a plurality of grooves defined by the portions of the elongate body 24 that are formed around each contact 32. The electrically insulative material of the elongate body 24 protects the strut 34 and the conductors 38 while also allowing for electrical communication between the contacts 32 and a corresponding header. Additionally, because portions of the elongate body 24 are formed between and separate each contact 32 from an adjacent contact 32, the potential for electrical interference between multiple contacts 32 is reduced.

In an exemplary embodiment, each electrical contact 32 may be a “ring” shaped contact. However, the electrical contacts 32 may have any other shape or configuration deemed appropriate to correspond to a respective header such as, but not limited to: linear, curved, zig-zag, or round. Each electrical contact 32 may be spaced apart from one another along the longitudinal axis of the elongate body 24 such that there is sufficient distance between each adjacent electrical contact 32 to reduce the risk of electrical interference between contacts. Although, each electrical contact 32 may be equally spaced apart from its adjacent electrical contact, the electrical contacts 32 may also be unequally spaced apart such that a distance between a first pair of electrical contacts is greater than or less than the distance between a second pair of electrical contacts.

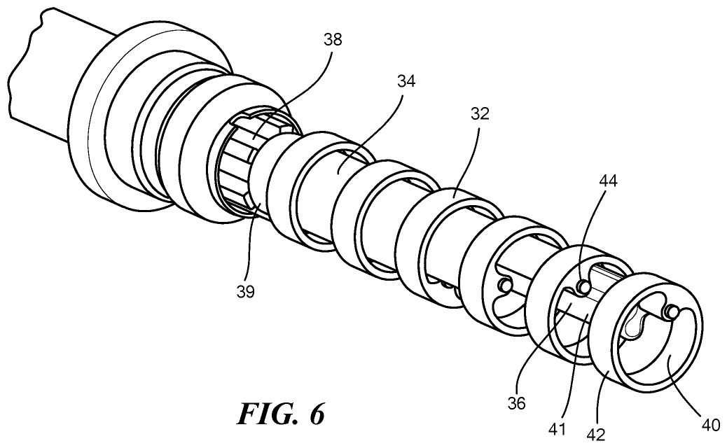

As shown in more detail in FIG. 6, each contact 32 may have an inner portion 40 and an exterior portion 42. The interior portion 40 of each contact 32 defines a receiving member or aperture 44 sized to receive a distal end of one of the conductors 38 within the strut channels 36. The insertion of the distal ends of the conductors 38 into the apertures 44 enables electrical communication between the contacts 32 and the conductors 38. The electrical communication of the electrical contacts 32 and the conductors 38 allows for electrical current to be delivered from the controller 14 to the contacts 32, whereas the physical connection of the conductors 38 to the contacts 32 further contributes to restricting the movement of the contacts 32. When the MSCD system is fully assembled, the connector 10 is inserted into a corresponding mating member, such as, for example, the connector header 37 (as shown in FIG. 3-4), that is electrically connected to the blood pump 16 so that each contact 32 is aligned and received within a corresponding groove within the header. The current delivered to the plurality of contacts 32 is then delivered to the blood pump 16 to provide the necessary power for operation of the pump 16.

In one embodiment, the connector 10 may include six electrical contacts 32 spaced apart on the elongate body 24 and six corresponding conductors 38 in communication with a respective electrical contact 32 via the channels 36 of the strut 34, thus providing six poles or contacts on a single connector. The connector 10 is not limited to having only six electrical contacts 32 and corresponding conductors 38, and thus may also include more than or fewer than six electrical contacts 32 and six conductors 38. For example, in one embodiment, the connector 10 may have at least two contacts 32 that are in communication with at least two conductors 38.

In one embodiment, the connector 10 also includes an insertion marker and a strain relief (not shown). The insertion marker may be a band, marker, or other type of visual indicator that is incorporated into or disposed on the outer surface 30 of the proximal end 26 of the connector 10. The insertion marker may be used to help a clinician identify the connection between the connector 10 and a corresponding header, and indicate full insertion when implanting the connector 10 in a patient during a surgical procedure. The band or marker may be color coded and/or marked to improve clinician visibility and/or usability. Further, the strain relief, may be disposed within the connector 10 at a position proximate to the proximal end 26 of the elongate body 24 and reduces stiffness within the transition regions of the connector 10. The strain relief may have a spiral, coil, helical, or any other configuration deemed appropriate to relieve tension within the connector 10.

In one embodiment, the elongate body 24 also includes an electrically insulative seal (not shown) disposed within the elongate body 24 between the proximal end 26 and the distal end 28 that allows for the electrical isolation of particular groupings of the conductors 38. For example, in an embodiment with six electrical contacts 32 extending between the proximal end 26 and distal end 28, the insulative seal may be disposed between the third and fourth electrical contacts 32 so that six contacts 32 are electrically separated into two isolated sets of contacts 32 that control different parts of the pump 16. As a non-limiting example, the two isolated sets of contacts 32 may include a first set of contacts 32, which may be the three contacts 32 nearest to the proximal end 26, and a second set of contacts 32, which may be the three contacts nearest to the distal end 28. The electrical isolation of the two sets of contacts 32 allows for the first and second sets of contacts 32 to each provide power from the controller 12 to different components or portions of the same pump 16. Additionally, the electrical contacts 32 may be grouped together in any other fashion desired. For example, the contacts 32 may be grouped in a manner so that the first set of contacts 32 includes the first, third, and fifth contacts 32 extending along the elongate body 24, and the second set of contacts 32 includes the second, fourth, and sixth contacts 32. Further, the contacts 32 may also be isolated into more than two groups. For example, the contacts 32 may be separated into sets of two so that there are three total sets of contacts 32 to provide power to three different portions of the pump 16. Although the seal allows for isolation and grouping of particular conductors 38 and contacts 32, the connector 10 may also be “keyed” or configured to allow for partial operation of the VAD if less than all of the conductors and contacts 32 are in use, regardless of whether the seal is present within the elongate body 24.

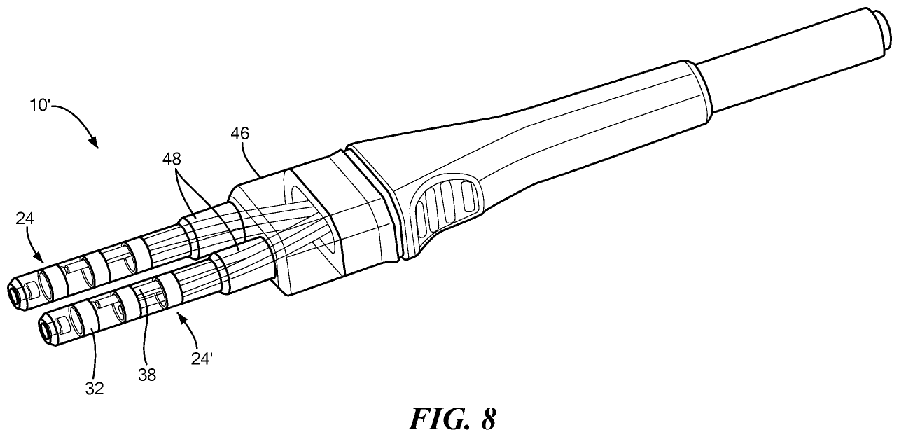

Now referring to FIG. 8, in one embodiment, the connector 10 may also be furcated so that the connector 10 includes more than one elongate body, as described herein, for use with a TETS system. The furcated connector, designated as 10′, may include the first elongate body 24 and a second elongate body 24′ parallel to the elongate body 24. The first elongate body 24 and second elongate body 24′ each partially extend within a bushing 46 of a connector body 48. The partial retention of the elongate bodies 24 and 24′ within the bushing 46 provides increased stability by providing a landing zone for the set screws 48 to minimize movement of the elongate bodies 24 and 24′. In this configuration, the furcated connector 10′ allows for the use of additional conductors 38 and electrical contacts 32, which divide the electrical current supplied by the battery 18 and allows for higher energy levels while maintaining low overall contact resistance between each parallel elongate body. The furcated connector 10′ is sized and configured so that each elongate body 24, 24′ is received within a single corresponding header, such as, for example, the header 37 shown in FIG. 3. The second elongate body 24′ may have the same length as the first elongate body 24, or it may also have a different length. The second elongate body 24′ may also have the same number of conductors 38 and contacts 32 as the first elongate body 24, or it may include a different number of conductors 38 and contacts 32 as the first elongate body 24.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A connector for a medical device, comprising:
an elongate body having a proximal end and a distal end;
a strut disposed within the elongate body, the strut defining a plurality of channels each having a proximal end and a distal end opposite the proximal end;
a plurality of conductors disposed within the elongate body, the plurality of conductors being sized and configured to be received within the plurality of channels and extend between the proximal end and the distal end of the elongate body; and
a plurality of electrical contacts coupled to an outer surface of the elongate body, the plurality of electrical contacts being in communication with the plurality of conductors.

2. The connector of claim 1, wherein the elongate body further defines a plurality of grooves, each of the plurality of electrical contacts being aligned and retained within a respective groove of the plurality of grooves.

3. The connector of claim 2, wherein the plurality of electrical contacts each have an inner portion and an outer portion opposite the inner portion, the inner portion defining an aperture sized to receive a distal end of one conductor from the plurality of conductors.

4. The connector of claim 3, wherein each channel of the plurality of channels progressively decreases in length between a longest channel of the plurality of channels and a shortest channel of the plurality of channels.

5. The connector of claim 4, wherein each of the plurality of electrical contacts are ring contacts.

6. The connector of claim 5, wherein the strut is composed of a first electrically insulative material.

7. The connector of claim 6, further including a non-conductive sealing surface disposed within the elongate body between the proximal end and the distal end.

8. The connector of claim 7, wherein the plurality of electrical contacts includes a first set of electrical contacts and a second set of electrical contacts, the non-conductive sealing surface being positioned between the first and second sets of electrical contacts to electrically isolate each respective set from one another.

9. The connector of claim 8, wherein the elongate body has an outer diameter between the range of 0.160 to 0.250 inches.

10. The connector of claim 9, wherein the elongate body defines a longitudinal axis between the proximal end and the distal end, each of the plurality of electrical contacts being spaced apart from an adjacent electrical contact along the longitudinal axis.

11. The connector of claim 10, wherein the elongate body is composed of a second electrically insulative material selected from the group consisting of polyurethane, polyethylene, urethane, and polyether ether ketone (PEEK).

12. A connector for a medical device, comprising:
a first elongate body;
a second elongate body parallel to the first elongate body, the first elongate body and the second elongate body each having a proximal end and a distal end opposite the proximal end;
a first plurality of conductors disposed within the first elongate body;
a second plurality of conductors disposed within the second elongate body;
a first plurality of electrical contacts extending around an outer surface of the first elongate body, the first plurality of conductors being in communication with the first plurality of electrical contacts; and
a second plurality of electrical contacts extending around an outer surface of the second elongate body, the second plurality of conductors being in communication with the second plurality of electrical contacts.

13. The connector of claim 12, wherein the first plurality of conductors includes more conductors than the second plurality of conductors

14. The connector of claim 12, wherein the first and second plurality of conductors each include at least two conductors.

15. The connector of claim 14, wherein the first and second plurality of electrical contacts each include at least two electrical contacts.

16. The connector of claim 15, wherein each electrical contact of the first and second plurality of electrical contacts is a ring contact.

17. The connector of claim 16, wherein the first elongate body and the second elongate body each define a longitudinal axis between the proximal end and the distal end, each ring contact being spaced apart from an adjacent ring contact along the longitudinal axis of the first elongate body and the second elongate body respectively.

18. The connector of claim 17, further including a non-conductive sealing surface disposed within at least one selected from the group consisting of the first elongate body and the second elongate body.

19. The connector of claim 18, wherein the first elongate body and the second elongate body are each made up of an electrically insulative material.

20. A connector for a medical device, comprising:
an electrically insulative elongate body having an outer diameter between the range of 0.160 to 0.250 inches, the electrically insulative elongate body including:
a proximal end;
a distal end opposite the proximal end;
a longitudinal axis defined between the proximal end and the distal end; and
a plurality of grooves defined by an outer surface of the electrically insulative elongate body along the longitudinal axis;
a strut disposed within the electrically insulative elongate body, the strut defining a plurality of channels each having:
a proximal end; and
a distal end opposite the proximal end, each channel of the plurality of channels progressively decreasing in length between a longest channel of the plurality of channels and a shortest channel of the plurality of channels; and
a plurality of conductors sized and configured to be received within the plurality of channels, the plurality of conductors including a first set of conductors and a second set of conductors;
a plurality of electrical ring contacts extending around the outer surface of the electrically insulative elongate body and retained within a respective groove of the plurality of grooves, each of the plurality of electrical ring contacts having an inner portion and an outer portion opposite the inner portion, the inner portion defining an aperture sized to receive a distal end of one conductor of the plurality of conductors; and
a non-conductive sealing surface disposed between the proximal end and the distal end of the elongate body, the non-conductive sealing surface being configured to electrically isolate the first set of conductors from the second set of conductors.
